(19) United States
(12) Patent Application Publication
(10) Pub. No.: US 2010/0186663 A1
(43) Pub. Date: Jul. 29, 2010

(54) METHODS AND APPARATUS FOR PROTECTING A SUBSTRATE SUPPORT IN A SEMICONDUCTOR PROCESS CHAMBER

(75) Inventor: Jon Christian FARR, Vallejo, CA (US)

Correspondence Address: MOSER IP LAW GROUP / APPLIED MATERIALS, INC., 1030 BROAD STREET, 2ND FLOOR, SHREWSBURY, NJ, 07702, US

(73) Assignee: APPLIED MATERIALS, INC., Santa Clara, CA (US)

(21) Appl. No.: 12/358,575

(22) Filed: Jan. 23, 2009

(51) Int. Cl. B05C 11/00; B08B 9/08; B08B 5/00; G01N 33/00

(52) U.S. Cl. 118/45; 134/22.1; 134/1.1; 73/865.9

(57) ABSTRACT

Methods and apparatus for protecting a substrate support in a semiconductor process chamber are provided herein. In some embodiments, an apparatus for protecting a substrate support in a semiconductor process chamber includes a semiconductor substrate and a coating comprising yttrium disposed on an upper surface of the semiconductor substrate. The semiconductor substrate is configured to be removably placed on the substrate support. In some embodiments, the semiconductor substrate comprises silicon. In some embodiments, the coating comprises yttrium and oxygen. In some embodiments, the protective apparatus may be disposed atop a substrate support within a semiconductor process chamber. In some embodiments, the protective apparatus may protect the surface of the substrate support from damage during one or more chamber processes, such as marathon testing or chamber cleaning.

### BACKGROUND

1. Field

Embodiments of the present invention generally relate to semiconductor process equipment, and more particularly, to an apparatus for protecting the surface of a substrate support during testing and cleaning processes.

2. Description of the Related Art

The processing environment of a semiconductor process chamber can lead to the corrosion and eventually failure of chamber components such as a substrate support. This failure can be accelerated during processes such as marathon testing of the process chamber or chamber cleaning processes wherein components of the process chamber are exposed to long cycle times or harsh process environments. During such processes, the support surface of the substrate support is typically protected by a semiconductor wafer. Unfortunately, semiconductor wafers are reactive to plasma and other such gases used during cleaning and/or testing, thereby undesirably resulting in a need to frequently stop the processes to replace the damaged wafers.

Thus, there is a need in the art for an improved apparatus to protect chamber components during processing over long cycle times.

### SUMMARY

Methods and apparatus for protecting a substrate support in a semiconductor process chamber are provided herein. In some embodiments, an apparatus for protecting a substrate support in a semiconductor process chamber includes a semiconductor substrate configured to be removably placed on the substrate support and a coating comprising yttrium disposed on an upper surface of the semiconductor substrate. In some embodiments, the semiconductor substrate comprises silicon. In some embodiments, the coating comprises yttrium and oxygen.

In some embodiments, a semiconductor processing system may include a process chamber having a substrate support disposed therein and a protective apparatus disposed on the substrate support. The protective apparatus may include a semiconductor substrate configured to be removably placed on the substrate support and a coating disposed on an upper surface of the semiconductor substrate and comprising yttrium.

In some embodiments, a method of performing a process in a semiconductor process chamber may includes disposing a protective apparatus atop a substrate support in the process chamber and performing a process in the semiconductor process chamber. The protective apparatus includes a semiconductor substrate configured to be removably placed on a substrate support having a coating comprising yttrium disposed on an upper surface of the semiconductor substrate.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts a process chamber having a coated silicon wafer in accordance with some embodiments of the present invention.

FIGS. 2A-B respectively depict schematic top and side views of a coated silicon wafer in accordance with some embodiments of the present invention.

FIG. 3 depicts a method for performing processes in a process chamber in accordance with some embodiments of the present invention.

To facilitate understanding, identical reference numerals have been used, where possible, to designate identical elements that are common to the figures. The figures are not drawn to scale and may be simplified for clarity. It is contemplated that elements and features of one embodiment may be beneficially incorporated in other embodiments without further recitation.

### DETAILED DESCRIPTION

Embodiments of the present invention provide methods and apparatus for performing processes within a semiconductor process chamber. The inventive methods and apparatus may advantageously facilitate efficient marathon testing and chamber cleaning/conditioning as compared to conventional methods and apparatus. The apparatus may include a substrate, such as a semiconductor wafer, having a coating comprising yttrium (Y). The coating may advantageously provide increased chemical resistivity to the process environment as compared with a bare silicon wafer. In some embodiments, the inventive apparatus and methods may be utilized in a process chamber of a substrate processing system, such as an etch chamber, a reactive ion etch (RIE) chamber, a chemical vapor deposition (CVD) chamber, a plasma enhanced CVD (PECVD) chamber, a physical vapor deposition (PVD) chamber, a thermal processing chamber, or the like. The inventive apparatus may be disposed atop a substrate support within a process chamber, and may protect a support surface of the substrate support during either marathon testing or cleaning of the process chamber. Thus, the inventive apparatus may facilitate repeated and continuous processing during marathon testing or cleaning of the process chamber as compared with conventional methods and apparatus.

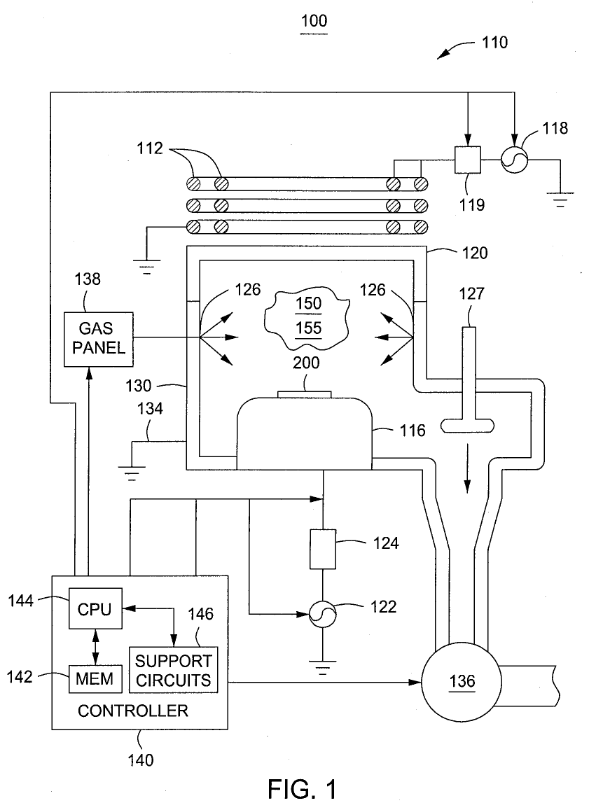

For example, FIG. 1 depicts a schematic diagram of an exemplary etch reactor 100 having a substrate support 116 with the inventive protective apparatus 200 disposed thereon. The reactor 100 includes a process chamber 110 having a conductive body (wall) 130, and a controller 140. The wall 130 defines an interior volume of the process chamber 110 having the substrate support 116 disposed therein. For the purposes of the present invention, the etch reactor 100 is merely an exemplary process chamber. Other suitable process chambers may be any chamber having one or more means for processing a substrate. Such means include etching, plasma etching, chemical vapor deposition, physical vapor deposition, any chamber capable of having a plasma or process gas disposed proximate the surface of a substrate support wherein the plasma or process gas is capable of damaging the substrate support, and the like.

The chamber 110 may be supplied with a dielectric ceiling 120. An antenna comprising at least one inductive coil element 112 may be disposed above the ceiling 120 (two co-axial elements 112 are shown). The inductive coil element 112 may be coupled through a first matching network 119 to a plasma power source 118.

The substrate support (cathode) 116 may be coupled through a second matching network 124 to a biasing power source 122. The biasing power may be either continuous or pulsed power. In other embodiments, the biasing power source 122 may be a DC or pulsed DC source.

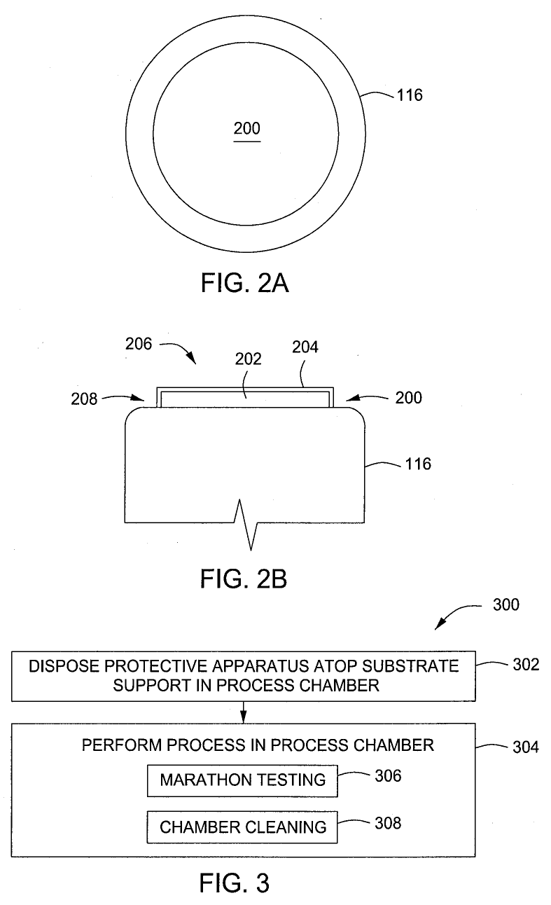

The protective apparatus 200 may be have a size and shape configured to be disposed on a support surface of the substrate support, as shown in FIG. 1. In some embodiments, the protective apparatus 200 may substantially cover the surface of the substrate support 116, as shown in FIGS. 2A-B, thereby protecting the surface of the substrate support 116 during certain processes where production substrates are not present, such as testing and cleaning of the process chamber 110. The protective apparatus 200 may protect the surface of the substrate support 116 from, for instance, corrosion or etching by prolonged exposure to a plasma 155 formed in the process chamber, or exposure to corrosive process gases in a non-plasma state.

As depicted in FIG. 2B, the protective apparatus 200 generally comprises a substrate 202 having a coating 204 disposed thereover. The substrate 202 may be any substrate suitable for processing in a semiconductor process chamber. As such, the protective apparatus 200 can advantageously have all the properties of a substrate typically processed by the process chamber 100, such as, for example, wafer handling, environmental conditions, electrostatic chucking, thermal behavior, backside surface conditions, and the like. Examples of suitable substrates include <111> and <110> silicon, silicon germanium, silicon carbon, doped silicon, and the like. Substrates having a diameter of 150, 200 or 300 mm may be typically used. However, substrates having any size capable of being held on the surface of the substrate support may be used. In some embodiments, such as illustrated in FIG. 2B, the semiconductor substrate 202 may be a single layer, for example, comprising silicon (Si). In some embodiments, the substrate 202 may comprise silicon (Si) having a layer of silicon oxide (SiO₂) disposed thereon (not shown).

In some embodiments, an upper surface 206 of the substrate 202 may be covered with the coating 204. In some embodiments, an edge of the substrate may be masked to provide an edge bead on the upper surface 206 that is not coated. In some embodiments, both the upper surface 206 and any other surface immediately exposed to the processing environment, such as edges 208, may be sheathed with the coating 204. In general, any surface of the substrate 202 may be covered with the coating 204 to limit the degradation of the substrate 202 in the process environment, and thus protect the surface of the substrate support from damage.

In some embodiments, a backside of the substrate 202 may be left bare, or uncoated. Leaving the backside of the substrate uncoated advantageously provides a standard substrate surface for interfacing with process system components, such as transfer robot blades, electrostatic chucks or substrate supports, and the like. Thus, thermal and electrical properties of the substrate will be the same as for a standard substrate, such as a silicon wafer. Thus, process testing may be performed with no need for adjustments when moving to product substrates.

The coating 204 comprises a non-reactive material, such as yttrium (Y), that can withstand corrosive process conditions in the process chamber 110. In some embodiments, the coating 204 comprises yttrium (Y) and oxygen (O₂). In some embodiments, the coating 204 may comprise alumina (Al₂O₃), although alumina is reactive with some process gases, such as fluorine. The coating 204 may be formed to a desired thickness and density on the substrate 202. In some embodiments, the coating 204 may be up to 1 mm thick. In some embodiments, the coating 204 may be between about 50 to about 100 microns thick. Thinner coatings may advantageously limit capacitive reactions when the protective apparatus 200 is utilized in a process chamber.

The non-reactive coating 204 facilitates processing in harsh environments repeatedly without the possibility of reactor contamination. In contrast, a conventional blanket oxide or bare silicon wafer has a very limited useful lifetime due to its reactivity with processing gasses. Thus, the protective apparatus 200 can be used repeatedly in cycling applications, thereby eliminating the need for interruption of the cycling process to change substrates (e.g., substrates with a thin layer of oxide currently used for process cycling and product testing have a limited useful lifetime and are required to be replaced once this layer of oxide is consumed by the reactor process). Thus, the protective apparatus 200 can be repeatedly used as a cover substrate for a substrate support in semiconductor wafer processing equipment with little or no reaction to the process to which it is exposed, thereby greatly reducing the need to change cover substrates for semiconductor wafer processing equipment.

The coating 204 may be applied in any suitable manner, such as by thermal plasma spray, or the like. Applying the coating 204 using thermal plasma spray processing may advantageously facilitate depositing a thicker coating on the substrate 202. Thermal plasma spray may further advantageously facilitate greater uniformity of the coating 204. Thermal plasma spray coating may further be more cost effective and less time consuming than other methods of depositing non-reactive materials onto silicon substrates.

Prior to coating the substrate 202, surfaces of the substrate desired to be coated may be modified to ensure robust adhesion between the substrate 202 and the coating 204. Suitable modifications can include roughening by sanding, blasting, chemical or mechanical etching, or the like. In some embodiments, the surface roughness may be selected in accordance with the type of powder spray being used. In some embodiments, the surface roughness of the substrate 202 may be up to about 100 Ra, or greater than 100 Ra. In some embodiments, the surface roughness may be a mirror finish (e.g., about 0 Ra). In some embodiments, the surface modification provides a surface finish of between about 125 and about 5 rms. In some embodiments, the coating 204 may be polished to reduce roughness after application to the substrate 202.

The substrate 202 may be pre-heated or situated on a warm surface prior to applying the coating 204. The pre-heating may be to between about 70 to about 300 degrees Celsius. Pre-heating the substrate 202 may be advantageous to limit thermal shock during formation of the coating 204, for example, via a thermal plasma spray process. Thermal shock may cause surface cracks in the substrate 202, or the coating 204, and thus undesirably expose the substrate 202 to the processing environment during marathon testing or chamber cleaning. After application of the coating 204, the substrate 202 may be cooled gradually to prevent cracks in the coating 204 and/or substrate 202. In some embodiments, the substrate 202 may be cooled at a rate of between about 0.5 to about 10 degrees Celsius/minute.

To facilitate control of the process chamber 110, as described below, the controller 140 may be one of any form of general-purpose computer processor that can be used in an industrial setting for controlling various chambers and sub-processors. The controller 140 may includes a memory 142, or computer-readable medium, a CPU 144, and support circuits 146. The memory 142 may be one or more of readily available memory such as random access memory (RAM), read only memory (ROM), floppy disk, hard disk, or any other form of digital storage, local or remote. The support circuits 146 are coupled to the CPU 144 for supporting the processor in a conventional manner. These circuits include cache, power supplies, clock circuits, input/output circuitry and subsystems, and the like. The inventive methods for controlling the apparatus as described herein may generally stored in the memory 142 as a software routine, which, when executed, may control the etch reactor 100 to perform the inventive methods. The software routine may also be stored and/or executed by a second CPU (not shown) that is remotely located from the hardware being controlled by the CPU 144.

In some embodiments, in operation, the protective apparatus 200 may be placed on the substrate support 116 and process gases supplied from a gas panel 138 through entry ports 126 to form a gaseous mixture 150 within the chamber 110. The gaseous mixture 150 may be excited into a plasma 155 in the chamber 110 by applying power from the plasma source 118 and biasing power source 122 to the inductive coil element 112 and the cathode 116, respectively. The pressure within the interior of the chamber 110 may be controlled using a throttle valve 127 and a vacuum pump 136. Typically, the chamber wall 130 is coupled to an electrical ground 134. The temperature of the wall 130 may be controlled using liquid-containing conduits (not shown) that run through the wall 130.

The protective apparatus 200 may be provided atop the substrate support 116 in the process chamber 110 during, for example, cycle testing and/or chamber cleaning to protect the substrate support from damage during such processing. For example, FIG. 3 depicts a flow chart of a method 300 in accordance with some embodiments of the present invention. The method 300 generally begins at 302, where the protective apparatus 200 may be disposed atop the substrate support 116 in the process chamber 110.

Next, at 304, a desired process may be performed in the process chamber 110. Generally, such processes may include any non-production process involving the flowing of process gases, gas mixtures, forming plasmas, or the like in proximity to the surface of the substrate support 116, and capable of damaging the surface of the substrate support 116, where a production substrate would typically not be present.

For example, in some embodiments, the processes performed in a process chamber may include cycle or marathon testing (306) and/or conditioning or cleaning (308) of the process chamber 110. Other processes may be performed as well where the protective apparatus 200 may be provided to protect the surface of the substrate support 116. For example, generally, processes performed in a process chamber including repeated or prolonged exposure of the substrate support to process gases, gas mixtures, plasmas, or the like, may be performed using the protective apparatus 200 to protect the substrate support. In some embodiments, processes may include harsh processing environments necessary to remove contaminants from one or more surfaces within a process chamber. Thus, the inventive protective apparatus 200 may be utilized during such processes to protect the surface of the substrate support.

In some embodiments, such as marathon testing, it may be required to repeatedly and continuously test one or more functions of a process chamber. For instance, and referring to the process chamber 110, it is contemplated that it may be required to repeatedly form the plasma 155 over several process cycles, or alternatively for continuous periods of time, as a means of assessing chamber performance and/or seasoning a process chamber. In some embodiments, a marathon test may be performed continuously for up to 1000 hours as a means of testing the performance of a process chamber. In some embodiments, a plasma may be formed from one or more process gases, or process gas mixtures including hydrogen bromide (HBr), nitrogen trifluoride (NF₃) and oxygen (O₂), silicon hexafluoride (SF₆), chlorine (Cl₂), nitrogen (N₂) and oxygen (O₂), silicon hexafluoride (SiF₆) and octafluorocyclobutane (C₄F₈), trifluoromethane (CHF₃), and other silicon and oxide etch chemistries.

In some embodiments, such as chamber cleaning and/or conditioning, it may be required to expose an interior volume of a process chamber to a harsh processing environment necessary for removing contaminants formed during the processing of a semiconductor wafer. It is contemplated that a chamber clean may be performed between the exchange of each substrate, after the processing of several substrates in a process chamber, or as desired or needed. The chamber cleaning process may include forming a plasma necessary to remove contaminants from a chamber wall and/or other surfaces of the process chamber. In some embodiments, a plasma may be formed from one or more process gases, or process gas mixtures, for example, for chamber cleaning and polymer removal (e.g., oxygen (O₂) and the like), for chamber cleaning (e.g., fluorine-based chemistries, such as nitrogen trifluoride (NF₃), or chlorine-based cleaning chemistries, and the like), and/or for chamber conditioning (e.g., silicon hexafluoride (SiF₆) and octafluorocyclobutane (C₄F₈) and the like).

Thus, methods and apparatus for protecting a substrate support in a semiconductor process chamber are provided herein. In some embodiments, a protective apparatus may be provided atop a substrate support during chamber processing such as marathon testing, cleaning, and/or conditioning of the process chamber. The protective apparatus may advantageously protect the surface of the substrate support from harsh processing environments and/or prolonged exposure to one or more process gases, gas mixtures, and/or plasmas formed during such processing. The protective apparatus, may advantageously provide a long life

While the foregoing is directed to embodiments of the present invention, other and further embodiments of the invention may be devised without departing from the basic scope thereof, and the scope thereof is determined by the claims that follow.

What is claimed is:

1. An apparatus for protecting a substrate support in a semiconductor process chamber, comprising:
a semiconductor substrate configured to be removably placed on a substrate support; and
a coating comprising yttrium (Y) disposed on an upper surface of the semiconductor substrate.

2. The apparatus of claim 1, wherein the semiconductor substrate comprises at least silicon (Si), silicon oxide (SiO₂) or a combination thereof.

3. The apparatus of claim 1, wherein the coating further comprises oxygen.

4. The apparatus of claim 1, wherein the semiconductor substrate comprises silicon and the coating comprises yttrium and oxygen.

5. The apparatus of claim 1, the semiconductor substrate comprises silicon and oxygen and the coating comprising yttrium and oxygen.

6. The apparatus of claim 1, wherein the coating is further disposed on an edge of the semiconductor substrate.

7. The apparatus of claim 1, wherein the semiconductor substrate comprises one of a 150, 200, or 300 mm semiconductor wafer.

8. A semiconductor processing system, comprising:
a process chamber having a substrate support disposed therein; and
a protective apparatus disposed on the substrate support, the protective apparatus comprising:
a semiconductor substrate configured to be removably placed on a substrate support; and
a coating disposed on an upper surface of the semiconductor substrate and comprising yttrium (Y).

9. The system of claim 8, further comprising:
an RF generator coupled to the process chamber for forming a plasma therein.

10. The system of claim 8, the semiconductor substrate comprising at least one of silicon (Si) or silicon oxide (SiO₂).

11. The system of claim 8, wherein the coating further comprises oxygen.

12. The apparatus of claim 8, wherein the semiconductor substrate comprises silicon and the coating comprises yttrium and oxygen.

13. The apparatus of claim 8, the semiconductor substrate comprises silicon and oxygen and the coating comprising yttrium and oxygen.

14. The apparatus of claim 8, wherein the coating is further disposed on an edge of the semiconductor substrate.

15. A method of performing a process in a semiconductor process chamber, comprising:
disposing a protective apparatus atop a substrate support in the process chamber, wherein the protective apparatus comprises a semiconductor substrate having a coating comprising yttrium (Y) disposed on an upper surface of the semiconductor substrate; and
performing a process in the semiconductor process chamber.

16. The method of claim 15, wherein the process comprises marathon testing.

17. The method of claim 15, wherein the process comprises a chamber clean process.

18. The method of claim 15, wherein the process comprises forming a plasma in the process chamber.
